(19) United States
(12) Patent Application Publication
(10) Pub. No.: US 2024/0286110 A1
(43) Pub. Date: Aug. 29, 2024

(54) CARBON MICROSPHERE CHROMATOGRAPHIC MATERIAL

(71) Applicant: Chrom6 Technologies, LLC, Salt Lake City, UT (US)

(72) Inventor: Andrew E. Dadson, Vineyard, UT (US)

(21) Appl. No.: 18/586,453

(22) Filed: Feb. 24, 2024

Related U.S. Application Data
(60) Provisional application No. 63/448,269, filed on Feb. 25, 2023.

(51) Int. Cl. B01J 20/20; B01D 15/08; B01J 20/22; B01J 20/28; B01J 20/282; B01J 20/30; B01J 20/32; C07C 7/10; C07C 51/47; C07C 67/56; C07C 231/24

(52) U.S. Cl. CPC B01J 20/20; B01D 15/08; B01J 20/22; B01J 20/28004; B01J 20/28019; B01J 20/28059; B01J 20/28061; B01J 20/282; B01J 20/3078; B01J 20/3204; B01J 20/3219; B01J 20/3246; B01J 20/3293; C07C 7/10; C07C 51/47; C07C 67/56; C07C 231/24; C07B 2200/09

(57) ABSTRACT

A chromatographic material of carbon microspheres is disclosed. The carbon microspheres may be surface-derivatized with an organic functional group, such as functional groups suitable for use in chromatographic process, such as C1 to C18 alkyl, including butyl (C4), octyl (C8), and octadecyl (C18), phenyl, amino, cyano, etc. The carbon microspheres may consist of nonporous carbon. The carbon microspheres may consist of porous carbon having a surface area in the range of 1 m²/g to 400 m²/g. A method of making carbon microspheres from a carbonizable polymeric material is disclosed. A method of bonding an organic functional group to a surface of the carbon microspheres is disclosed. HPLC columns may be prepared containing the chromatographic material of carbon microspheres. Such columns may be used to separate a compound of interest from a mixture of different compounds using a chromatographic process, such as HPLC.

### CROSS-REFERENCE TO RELATED APPLICATION

This application claims the benefit of U.S. Provisional Application No. 63/448,269 filed Feb. 25, 2023, titled CARBON MICROSPHERE CHROMATOGRAPHIC MATERIAL, which application is incorporated by reference.

### FIELD OF THE INVENTION

The presently disclosed invention provides novel chromatographic materials, e.g., for chromatographic separations, processes for its preparation and separation devices containing the chromatographic material. The present disclosure relates more particularly in various embodiments, to carbon microsphere chromatographic materials for liquid chromatography (LC), particularly high-performance liquid chromatography (HPLC), and Solid Phase Microextraction (SPME) on both analytical and preparatory scales as well as corresponding apparatuses, kits, methods of manufacture, and methods of use.

### BACKGROUND OF THE INVENTION

Chromatography is the collective term for a set of laboratory techniques for the separation, quantitation, and identification of the components of a mixture. The mixture is dissolved in a mobile phase, which carries it through a packed column containing a packed bed with a stationary phase. The various constituents of the mixture interact differently with the stationary phase, causing them to separate. The separation is based on differential partitioning between the mobile and stationary phases. Subtle differences in a compound's partition coefficient result in differential retention on the stationary phase, thus changing the separation. Chromatography is useful for the separation of compounds that are structurally related, such as stereoisomers, chiral compounds, diastereomers, etc. Some techniques, including supercritical fluid chromatography (SFC), are known for being particularly useful for separating structurally related vitamins, natural products and chemical materials. Often, however, chromatographic techniques are insufficient to separate all structurally related compounds. For example, critical pairs of related vitamins (e.g., D2 and D3, K1 and K2) are difficult to separate/resolve. It is common to combine chromatography with an identification technique such as Mass Spectrometry. This technique is usually abbreviated as LC-MS and is particularly useful in analyzing difficult to separate mixtures.

Mobile phases are often solvent-based liquids, although gas chromatography typically employs gaseous mobile phases. Liquid mobile phases may vary significantly in their compositions depending on various characteristics of the sample being analyzed and on the various components sought to be extracted and/or analyzed in the sample. For example, liquid mobile phases may vary significantly in pH and solvent properties. Additionally, liquid mobile phases may vary in their compositions depending on the characteristics of the stationary phase that is being employed. Often, two or more different mobile phases are employed during a given chromatography procedure. Stationary phase materials may also exhibit poor stability characteristics in the presence of various mobile phase compositions and/or complex mixtures for which separation is desired. The poor stability characteristics of stationary phase materials in some mobile phases and complex mixtures, in some cases, may even preclude the possibility of using chromatography to perform the desired separation.

Packing materials for liquid chromatography can be generally classified into two types: organic materials, for example, polydivinylbenzene, and inorganic materials, for example, silica. Hybrid packing materials have also been developed which combine characteristics of organic and inorganic materials. Many organic materials are chemically stable against strongly alkaline and strongly acidic mobile phases, allowing flexibility in the choice of mobile phase composition and pH. However, organic chromatographic materials can result in columns with low efficiency, particularly with low molecular-weight analytes. Many organic chromatographic materials not only lack the mechanical strength of typical chromatographic silica and also shrink and swell when the composition of the mobile phase is changed.

Silica is widely used in high-performance liquid chromatography (HPLC), ultra-high performance liquid chromatography (UHPLC), and supercritical fluid chromatography (SFC). Some applications employ silica that has been surface-derivatized with an organic functional group such as octadecyl (C18), octyl (C8), phenyl, amino, cyano, and the like. As stationary phases for HPLC, these packing materials can result in columns that have high efficiency and do not show evidence of shrinking or swelling.

There remains a need for alternative materials that provide additional mechanical strength, increased column efficiency, and chromatographic selectivity.

### SUMMARY OF THE INVENTION

The present disclosure relates to carbon microsphere chromatographic materials. One aspect of the present disclosure relates to a chromatographic material of carbon microspheres which are surface-derivatized with an organic functional group. One aspect of the present disclosure relates to a chromatographic material of nonporous carbon microspheres.

Implementations of the chromatographic material may include one or more of the following features. Non-limiting examples of organic functional group include alkyl, phenyl, amino, and cyano. Alkyl organic functional groups may range in size from C1 to C18. The alkyl organic functional group may be selected from butyl (C4), octyl (C8), or octadecyl (C18). Other non-limiting examples of organic functional groups include pentafluorophenyl, mixed-mode chromatography ligands selected to provide multiple types of interactions such as affinity, hydrophobic interaction (HIC), and cation/anion exchange, and charged species suitable for anionic and cationic exchange separations. The carbon microspheres may be stable at a temperature in the range from about 25° C. (ambient temperature) to over 200° C. The carbon microspheres may be stable at a pH in the range from about 0 to 14. The carbon microspheres may be porous or nonporous. As used herein, the carbon microspheres are “stable” under a given use condition if the carbon microspheres remain mechanically robust and do not experience significant bleeding off of the stationary phase. The carbon microspheres may include solid carbon. The carbon microspheres may include a surface area less than 1 m²/g. The carbon microspheres may include a surface area less than 2 m²/g. The carbon microspheres may include a surface area less than 5 m²/g. The carbon microspheres may include a surface area less than 10 m²/g. The carbon microspheres may include a surface area in a range from 1 to 400 m²/g. The carbon microspheres may include a surface area in a range from 100 to 300 m²/g. The carbon microspheres may include a surface area of about 1, 2, 3, 4, 5, 6, 7, 8, 9, 10, 15, 20, 25, 30, 35, 40, 45, 50, 60, 70, 80, 90, 100, 110, 120, 130, 140, 150, 160, 170, 180, 190, 200, 225, 250, 275, 300, 325, 350, 375, or 400 m²/g, where any recited value may be an endpoint of a range.

Implementations of the chromatographic material may include one or more of the following features. The carbon microspheres may have a particle size less than 5 μm. The carbon microspheres may have a particle size less than 2 μm. The carbon microspheres may have a particle size less than 1 μm. The carbon microspheres may have a particle size in the range from about 2 μm to 5 μm. The carbon microspheres may have a particle size in the range from about 1 μm to 2 μm. The carbon microspheres may have a particle size greater than 10 μm.

One general aspect of the present disclosure relates to a chromatographic column having a stationary phase including a chromatographic material of carbon microspheres which are surface-derivatized with an organic functional group. One general aspect of the present disclosure relates to a chromatographic column having a stationary phase including a chromatographic material of nonporous carbon microspheres.

One general aspect of the present disclosure relates to a method of making a carbon microspheres. The method includes obtaining a plurality of microspheres of a carbonizable polymeric material. As used herein, carbonizable polymeric materials include, but are not limited to, polymeric materials which carbonize under the process steps disclosed herein. The method also includes oxidizing the polymeric microspheres in an oxygen-containing atmosphere at a temperature in the range of about 200° C. to 300° C. for a time period in the range of about 4 to 8 hours. The method also includes carbonizing the microspheres in an oxygen-free atmosphere by heating the microspheres to a temperature in the range of about 800° C. to 1000° C. over a time period of at least about 3 hours. The method also includes high temperature heating the microspheres in an oxygen-free atmosphere at a temperature greater than about 1200° C. for a time period of at least about 3 hours. The method also includes hydrogen treating the microspheres in an atmosphere which includes hydrogen (H₂) at a temperature in the range of about 900° C. to 1100° C. for a time period of at least about 1 hour to form a plurality of carbon microspheres. This hydrogenation step performs at least two functions: (1) it removes oxygen and heterogeneous species on the surface and (2) renders the surface uniform with hydrogen attachment points on the surface.

Implementations of the method of making a carbon microspheres may include one or more of the following features. The carbonizable polymeric material may include polystyrene divinylbenzene. The carbonizable polymeric material may include non-porous polystyrene divinylbenzene. The carbonizing oxygen-free atmosphere may include argon. The carbonizing oxygen-free atmosphere may include nitrogen. The carbonizing step is performed over a time period of at least about 4 hours. The microspheres are heated during the carbonizing step at a rate less than 5° C./min. The high temperature heating may be performed in an oxygen-free atmosphere comprising argon. The high temperature heating may be performed in oxygen-free atmosphere comprising nitrogen. The high temperature heating may be performed for a time period of at least about 4 hours. The high temperature heating may occur at a temperature in the range of about 1200° C. to 2500° C. The high temperature heating may occur at a temperature in the range of about 1500° C. to 2500° C. The high temperature heating may occur at a temperature in the range of about 2000° C. to 2500° C. The hydrogen heating may be performed for a time period of at least 2 hours.

Implementations of the method of making a carbon microspheres may include bonding an organic functional group to a surface of the carbon microspheres and may include one or more of the following features. The organic functional group may be selected to provide enhanced chromatographic selectivity, enhanced chromatographic column chemical stability, enhanced chromatographic column efficiency, and/or enhanced mechanical strength. The organic functional group may be selected from alkyl, phenyl, amino, and cyano. Alkyl organic functional groups may range in size from C1 to C18. The alkyl organic functional group may be selected from butyl (C4), octyl (C8), or octadecyl (C18). The organic functional group may include pentafluorophenyl, mixed-mode chromatography ligands selected to provide multiple types of interactions such as affinity, hydrophobic interaction (HIC), and cation/anion exchange, and charged species suitable for anionic and cationic exchange separations.

Implementations of the method of making a carbon microspheres may include bonding an organic functional group to a surface of the carbon microspheres by reacting the surface of the carbon microspheres with an iodide form of the organic functional group in the presence of lithium or sodium in ammonia.

One general aspect of the present disclosure relates to a method of separating a compound of interest from a mixture of compounds. The method includes providing the mixture containing the compound of interest. The method includes introducing a portion of the mixture to a chromatographic system having a chromatographic column. The method includes eluting the separated compound of interest from the chromatographic column. The chromatographic column has a stationary phase comprising a chromatographic material as disclosed herein.

This summary of the present invention is not intended to describe each illustrated embodiment or every possible implementation of the present invention. The figures and the detailed description that follow, however, do particularly exemplify these embodiments.

It is to be understood that both the foregoing general description and the following detailed description are exemplary and explanatory and are not restrictive of the invention, as claimed. It should be understood that the various embodiments are not limited to the arrangements and instrumentality shown in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIG. 1A is a graph of the HPLC separation results of a mixture of methyl paraben and ethyl paraben using a HPLC test column described in Example 4.

FIGS. 1B-1, 1B-2, and 1B-3 are graphs of the HPLC separation results, respectively, of a mixture of 2-methylhippuric acid, 3-methylhippuric acid, and 4-methylhippuric acid using a HPLC test column described in Example 4.

FIGS. 1C-1, 1C-2, and 1C-3 is a graph of the HPLC separation results of a mixture of o-, m-, and p-xylene using a HPLC test column described in Example 4.

FIG. 1D is a graph of the HPLC separation results of a mixture of methyl cis- and trans-2-hydroxycinnamic acid using a HPLC test column described in Example 4.

FIGS. 1E-1 and 1E-2 are graphs of the HPLC separation results of a mixture of Procainamide and prilocaine using a HPLC test column described in Example 4.

FIG. 2A is a graph of the HPLC separation results of a mixture of methyl paraben and ethyl paraben using a commercially available HPLC column described in Example 5.

FIGS. 2B-1, 2B-2, and 2B-3 are graphs of the HPLC separation results, respectively, of a mixture of 2-methylhippuric acid, 3-methylhippuric acid, and 4-methylhippuric acid using a commercially available HPLC column described in Example 5.

FIGS. 2C-1, 2C-2, and 2C-3 are graphs of the HPLC separation results, respectively, of a mixture of o-, m-, and p-xylene using a commercially available HPLC column described in Example 5.

FIG. 2D is a graph of the HPLC separation results of a mixture of methyl cis- and trans-2-hydroxycinnamic acid using a commercially available HPLC column described in Example 5.

FIGS. 2E-1 and 2E-2 are graphs of the HPLC separation results of a mixture of Procainamide and prilocaine using a commercially available HPLC column described in Example 5.

### DETAILED DESCRIPTION OF EMBODIMENTS

The present disclosure relates to carbon microsphere chromatographic materials. One aspect of the present disclosure relates to a chromatographic material of carbon microspheres which are surface-derivatized with an organic functional group. One aspect of the present disclosure relates to a chromatographic material of nonporous carbon microspheres.

One aspect of the present disclosure relates to a chromatographic column having a stationary phase including a chromatographic material of carbon microspheres which are surface-derivatized with an organic functional group. One aspect of the present disclosure relates to a chromatographic column having a stationary phase including a chromatographic material of nonporous carbon microspheres.

One aspect of the present disclosure relates to a method of making a carbon microspheres. The method includes obtaining a plurality of microspheres of a carbonizable polymeric material. The method also includes oxidizing the polymeric microspheres in an oxygen-containing atmosphere. The method also includes carbonizing the microspheres in an oxygen-free atmosphere by heating the microspheres to a sufficiently high temperature, such as about 800° C. to 1000° C. The method also includes high temperature heating the microspheres in an oxygen-free atmosphere. The method also includes hydrogen treating the microspheres in a high temperature atmosphere which includes hydrogen (H₂) to form a plurality of carbon microspheres.

One aspect of the present disclosure relates to a method of separating a compound of interest from a mixture of compounds. The method includes providing the mixture containing the compound of interest. The method includes introducing a portion of the mixture to a chromatographic system having a chromatographic column. The method includes eluting the separated compound of interest from the chromatographic column. The chromatographic column has a stationary phase comprising a carbon microsphere chromatographic material as disclosed herein.

Other features and advantages of the present invention are apparent from the different examples that follow. The examples below illustrate different aspects and embodiments of the present invention and how to make and practice them. The examples do not limit the claimed invention. Although methods and materials similar or equivalent to those described herein can be used in the practice of the present invention, suitable methods and materials are described below. Based on the present disclosure the skilled artisan can identify and employ other components and methodology useful for practicing the present invention.

### Example 1 Preparation of Carbon Microspheres

This example illustrates one process for preparing carbon microspheres. A plurality of nonporous polystyrene divinylbenzene microspheres having a particle size of 5 μm were oxidized in an oxygen-containing atmosphere at a temperature of 250° C. for a time period of about 6 hours. The particle size was measured by laser diffraction.

The oxidized microspheres were carbonized in a nitrogen atmosphere by heating, over a time period of about 4 hours, to a temperature of about 900° C. The microspheres were heated during the carbonizing step at a rate less than 5° C./min.

The carbonized microspheres were subjected to high temperature heating in an argon atmosphere at a temperature of 2400° C. for a time period of about 4 hours. The high temperature heat treatment densified the microspheres.

The microspheres were treated in a hydrogen (H₂) atmosphere at a temperature of 1000° C. for a time period of about 2 hours to form nonporous carbon microspheres. Without being bound by theory, this step performs at least two functions: (1) it removes oxygen and heterogeneous species on the surface and (2) renders the surface uniform with hydrogen attachment points on the surface. The carbon microspheres have a size of about 3.5 μm. The carbon microspheres have a surface area less than 1 m²/g. The carbon microspheres comprise solid carbon.

### Example 2 Bonding C4 Functional Groups to Carbon Microspheres

Butyl (C4) functional groups were bonded by reductive alkylation to the surface of nonporous carbon microspheres prepared according to Example 1. A plurality of the nonporous carbon microspheres were reacted with 1-iodobutane (C₄H₉I) in a mixture of lithium metal in condensed ammonia at −78° C.

The surface of the carbon microspheres was analyzed by attenuated total reflectance Fourier-transform infrared spectroscopy (ATR-FTIR), which confirmed the presence of C4 functionalized surface.

### Example 3 Bonding C18 Functional Groups to Carbon Microspheres

Octadecyl (C18) functional groups were bonded by reductive alkylation to the surface of nonporous carbon microspheres prepared according to Example 1. A plurality of the nonporous carbon microspheres were reacted with 1-iodooctadecane (C₁₈H₃₇I) in a mixture of lithium metal in condensed ammonia at −78° C.

The surface of the carbon microspheres was analyzed by attenuated total reflectance Fourier-transform infrared spectroscopy (ATR-FTIR), which confirmed the presence of C18 functionalized surface.

Example 4 Performance of HPLC column loaded with carbon microspheres functionalized with C4.

A 2.1×50 mm HPLC test column was prepared containing 3.5 μm nonporous carbon microspheres functionalized with C4 prepared according to the procedure of Example 3 as the stationary phase.

The test column was used to separate mixtures of the several different chemical species. The test column was operated at a temperature of 35° C. Solvent A consisted of 50 mL acetonitrile, 950 mL water, and 1 mL formic acid. Solvent B consisted of 800 mL acetonitrile, 200 mL water, and 1 mL formic acid. The flow rate was 0.3 mL/min. Detection was at UV 254 nm. Injection was 0.1 μL ca., 0.5 mg/mL in 20% methyl alcohol aqueous.

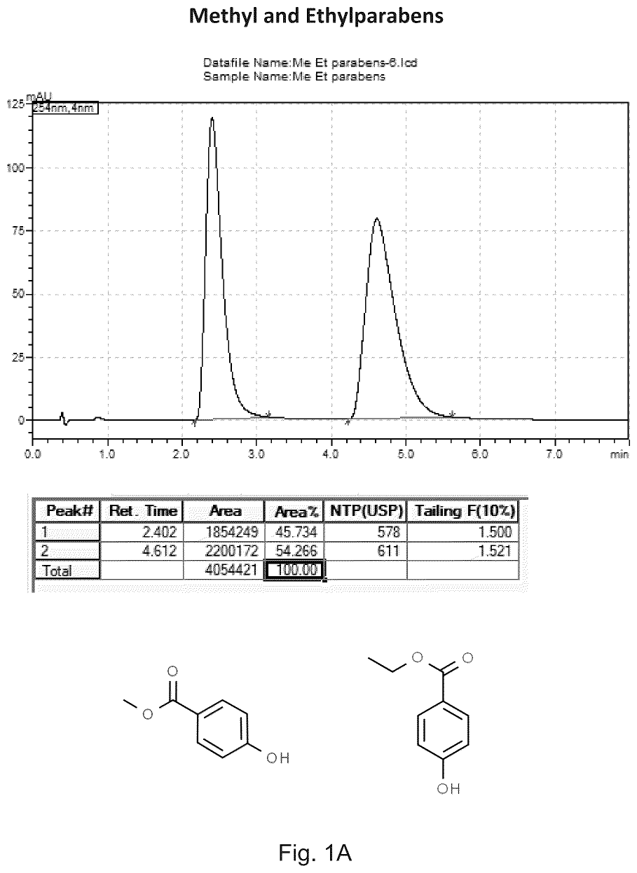

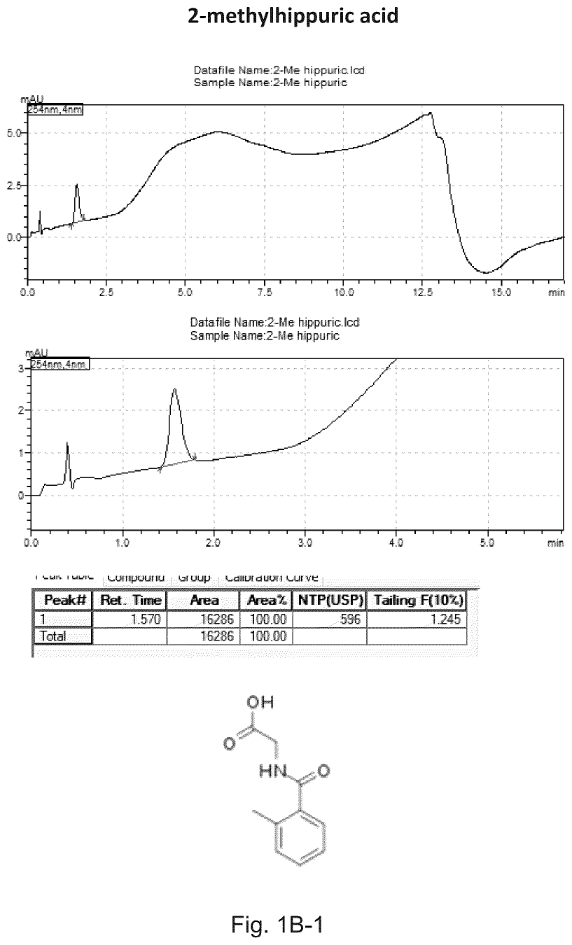

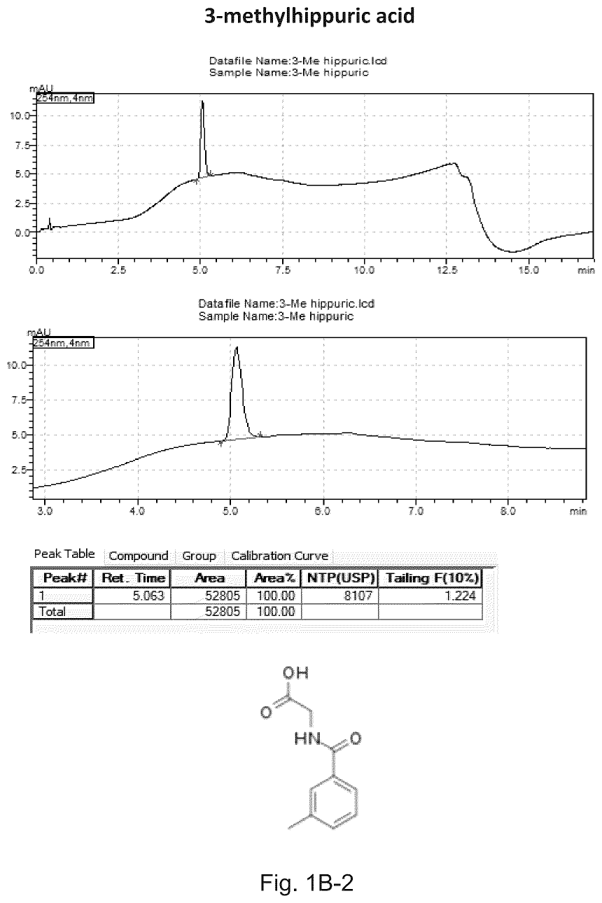

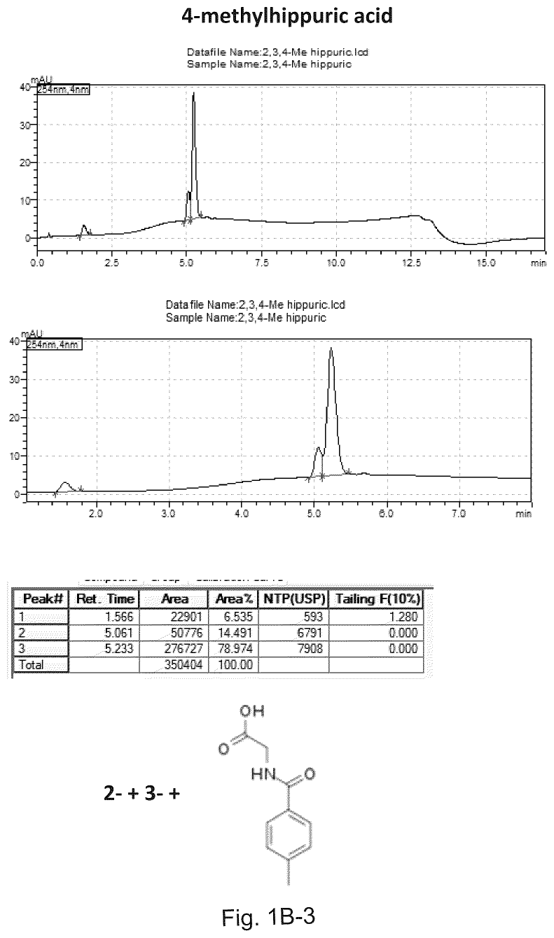

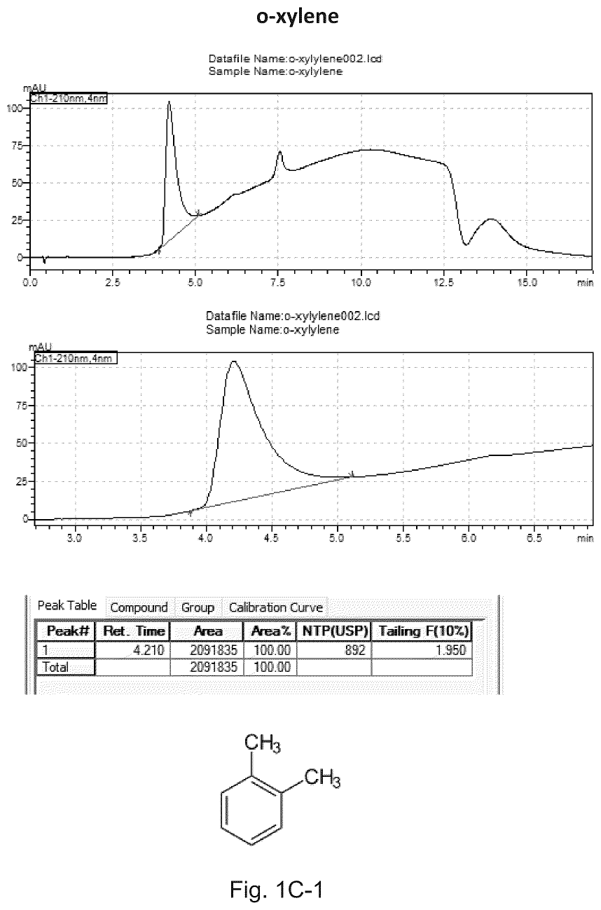

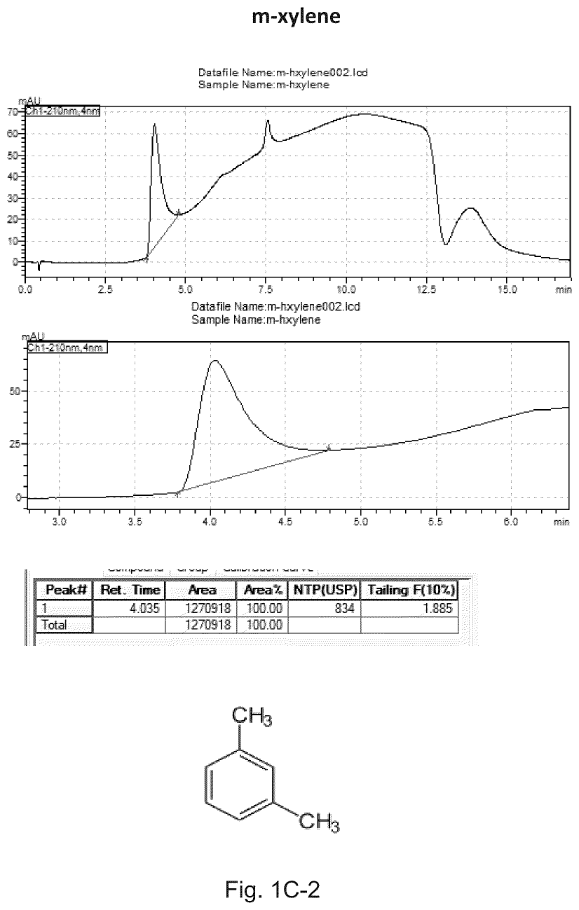

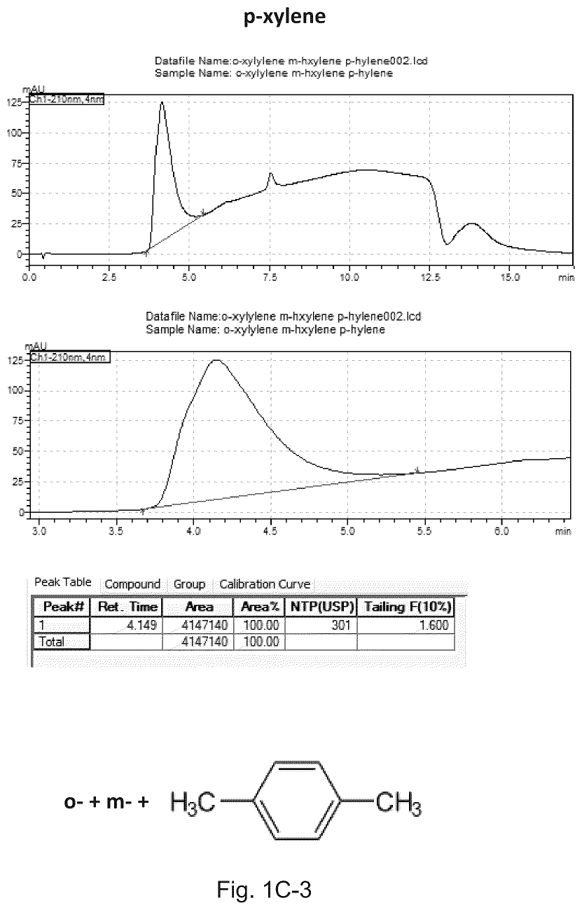

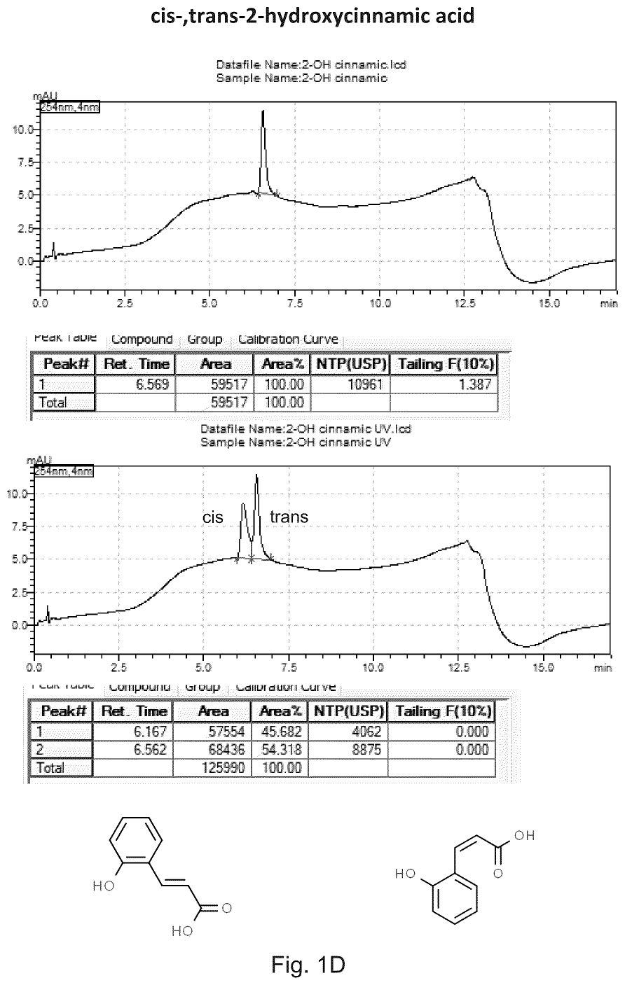

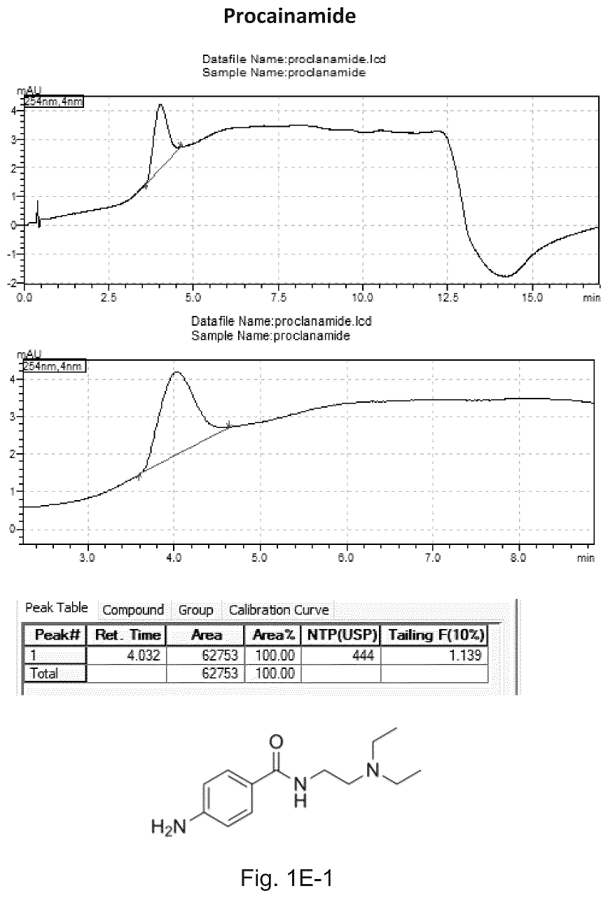

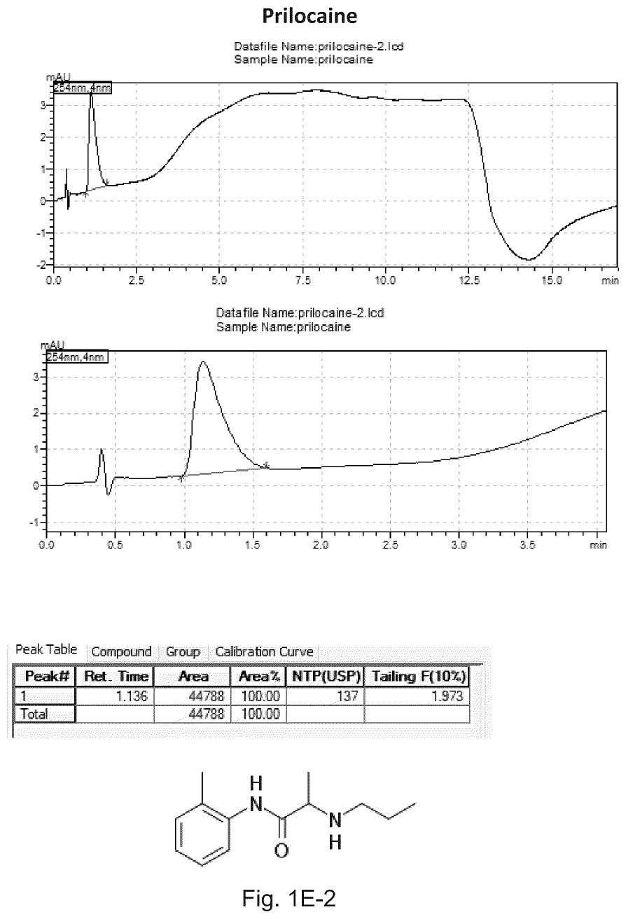

Methyl paraben and ethyl paraben were separated using an isocratic mixture of 75% solvent A and 25% solvent B. Performance results are shown in FIG. 1A. The results show clear baseline separation of these analytes.

2-, 3-, and 4-methylhippuric acid were separated using a gradient mixture of solvent A and solvent B according to Table 1. Performance results are shown in FIGS. 1B-1, 1B-2, and 1B-3. The results show resolution of these analytes.

TABLE 1

| Time (min) | % Solvent A | % Solvent B |
|---|---|---|
| 0.00 | 95 | 5 |
| 10.00 | 15 | 85 |
| 10.01 | 95 | 5 |
| 17.00 | 95 | 5 |

O-, m-, and p-xylene were separated using a gradient mixture of solvent A and solvent B according to Table 1. Performance results are shown in FIGS. 1C-1, 1C-2, and 1C-3. The results show resolution of these analytes.

Cis- and trans-2-hydroxycinnamic acid were separated using a gradient mixture of solvent A and solvent B according to Table 1. Performance results are shown in FIG. 1D. The results show resolution of these analytes.

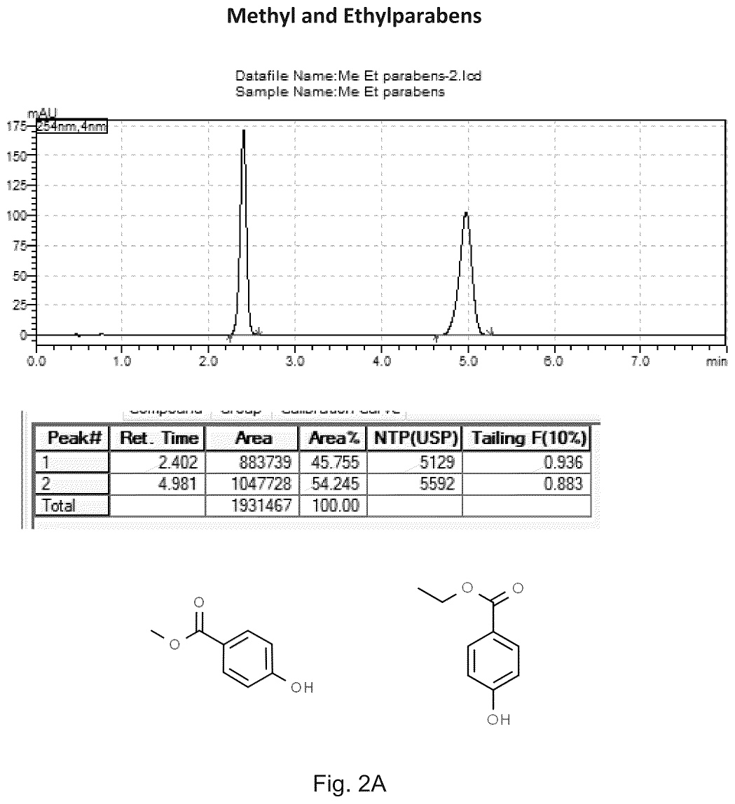

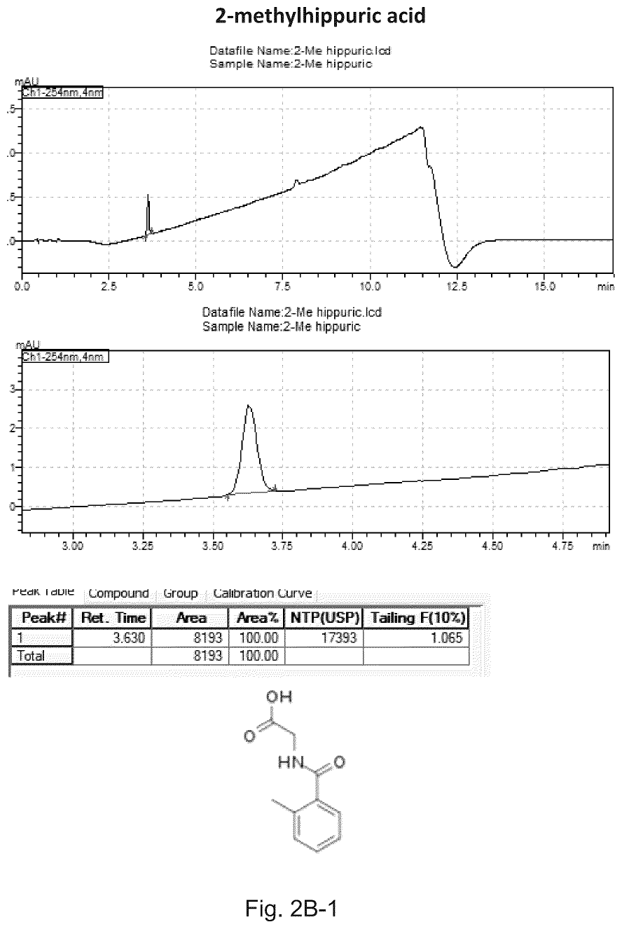

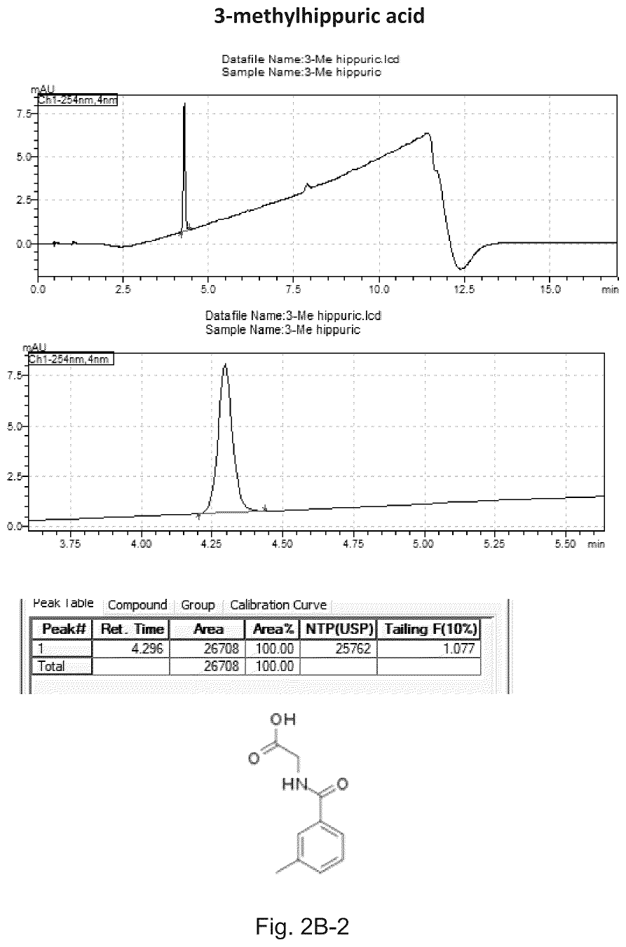

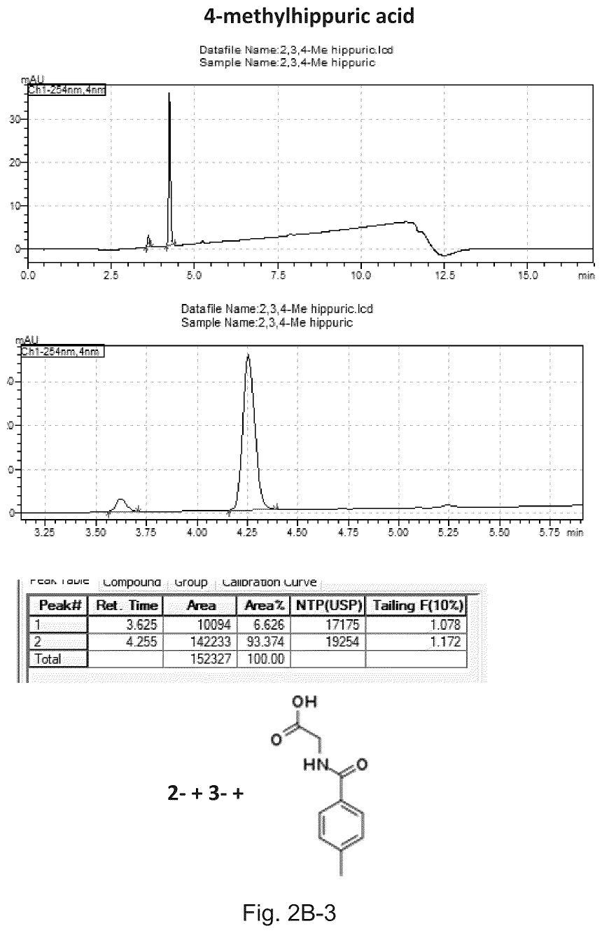

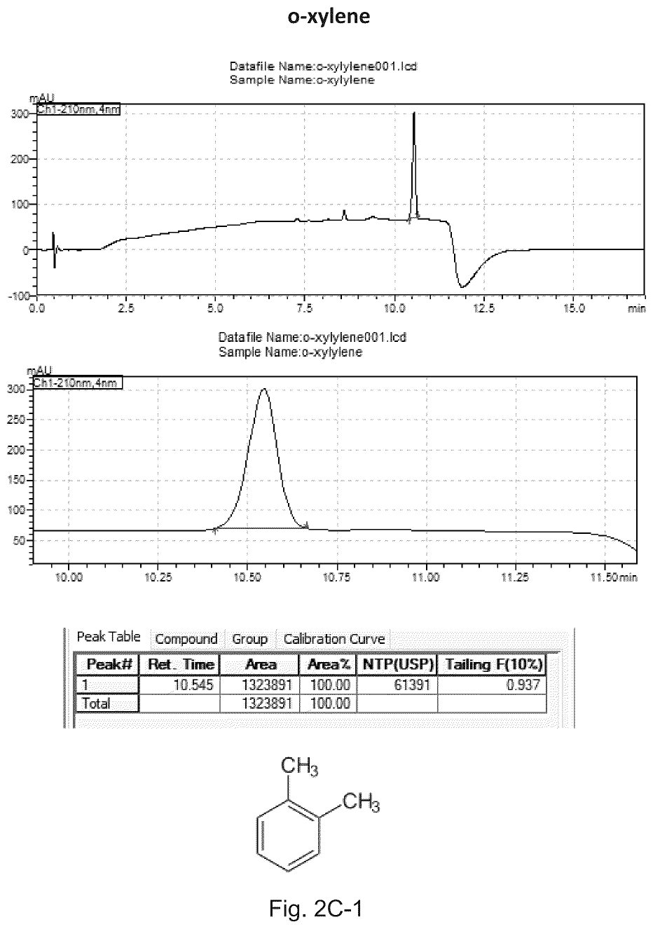

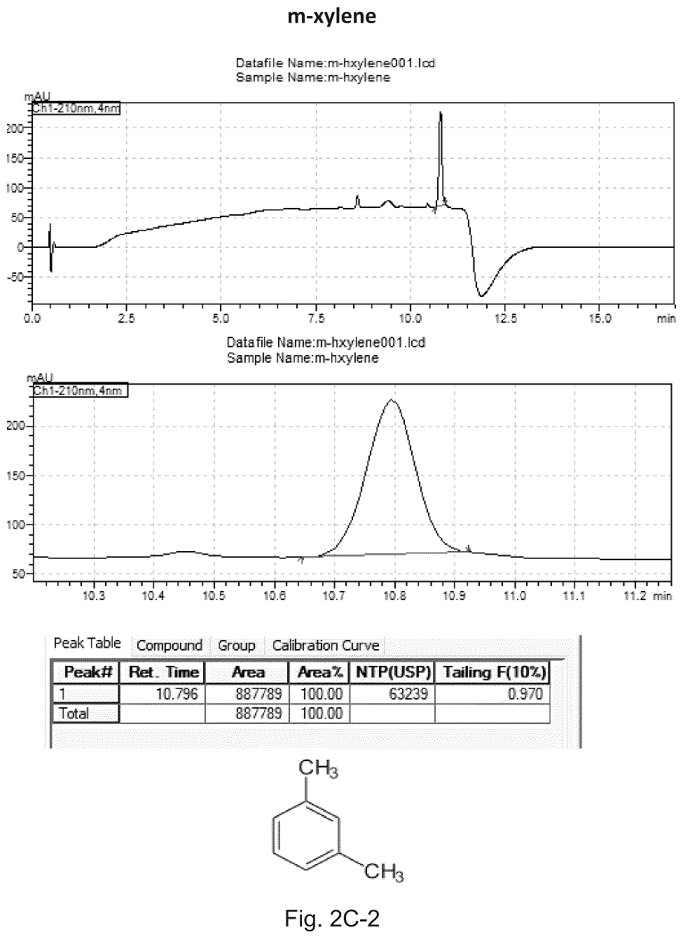

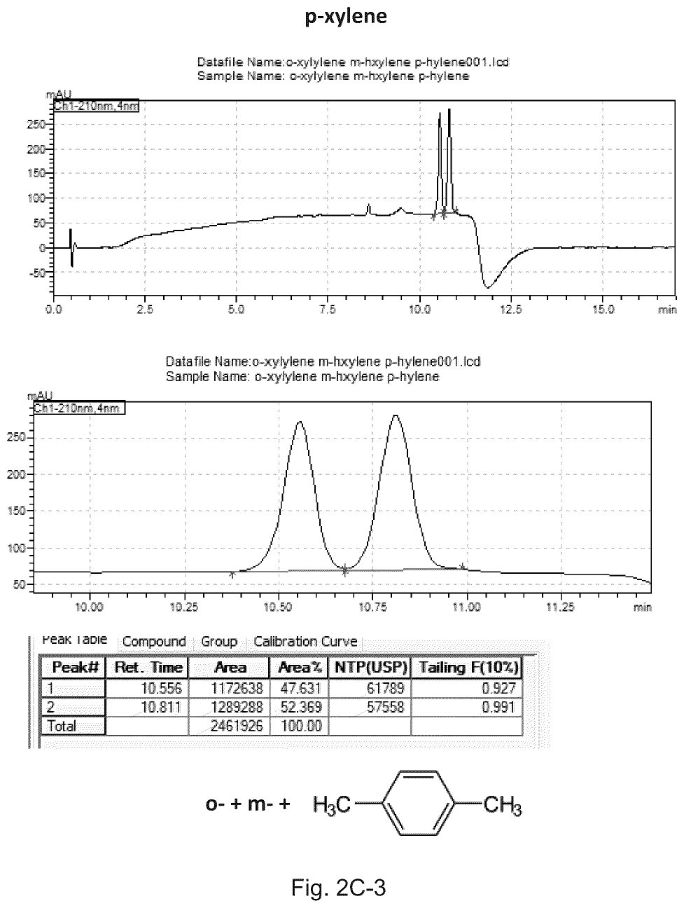

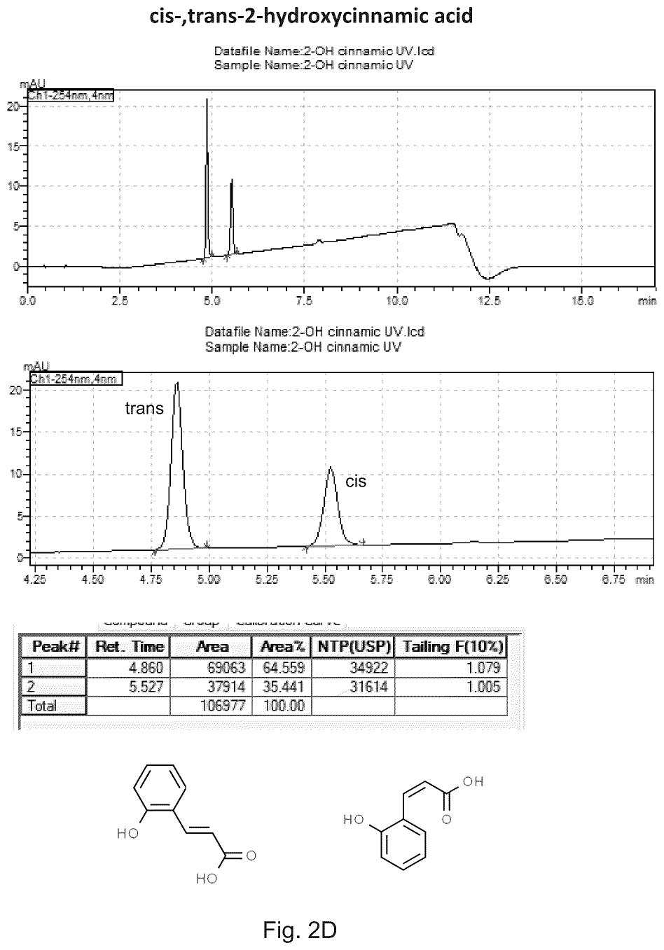

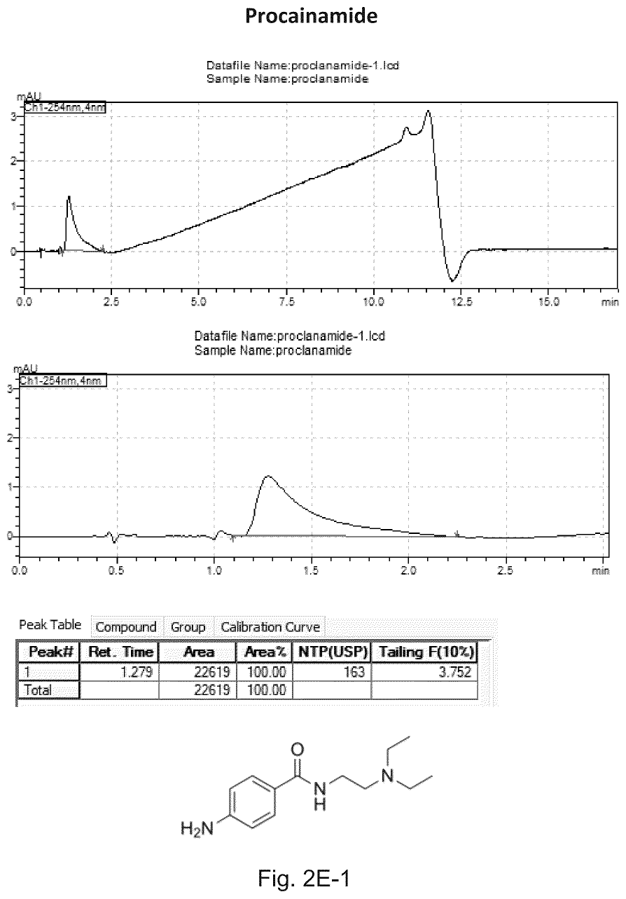

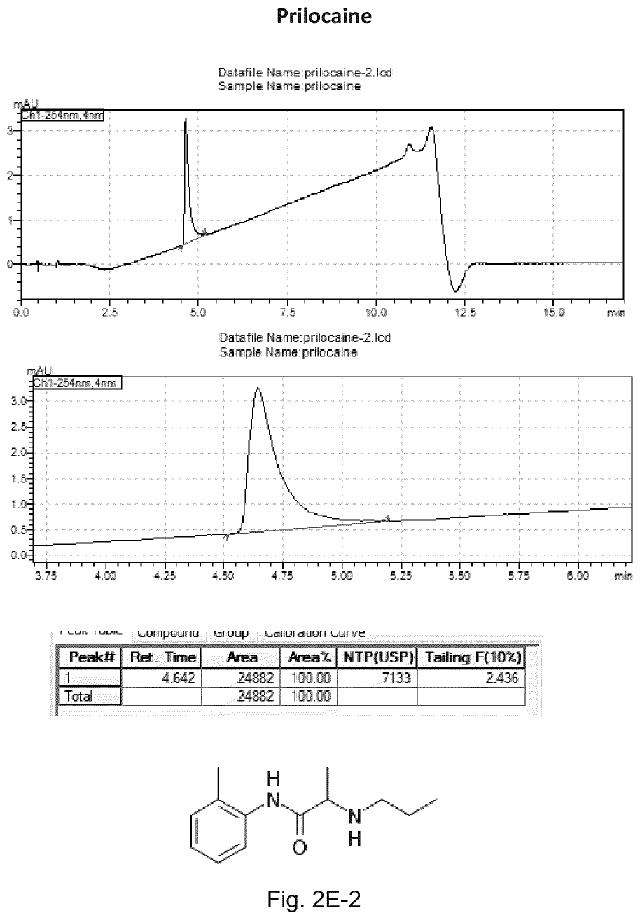

Procainamide and prilocaine were separated using a gradient mixture of solvent A and solvent B according to Table 2. Performance results are shown in FIGS. 1E-1 and 1E-2. The results show resolution of these analytes.

TABLE 2

| Time (min) | % Solvent A | % Solvent B |
|---|---|---|
| 0.00 | 95 | 5 |
| 10.00 | 50 | 50 |
| 10.01 | 95 | 5 |
| 17.00 | 95 | 5 |

The HPLC test column was able to effectively analyze different analyte types and demonstrated selectivity and the ability to separate closely related species on a short column.

### Example 5 Comparative Performance of Commercially Available HPLC Column Functionalized with C18

A commercially available HPLC column, ReproSil 70 C18 3 μm 3×50 mm, manufactured by Dr. Maisch, High Performance LC GmbH, was used to separate mixtures of the different chemical species identified in Example 4. The commercial column was operated at a temperature of 35° C. Solvent A consisted of 50 mL acetonitrile, 950 mL water, and 1 mL formic acid. Solvent B consisted of 800 mL acetonitrile, 200 mL water, and 1 mL formic acid. The flow rate was 0.6 mL/min. Detection was at UV 254 nm. Injection was 0.1 μL ca., 0.5 mg/mL in 20% methyl alcohol aqueous.

Methyl paraben and ethyl paraben were separated using an isocratic mixture of 75% solvent A and 25% solvent B. Performance results are shown in FIG. 2A. The results show clear baseline separation of these analytes.

2-, 3-, and 4-methylhippuric acid were separated using a gradient mixture of solvent A and solvent B according to Table 1. Performance results are shown in FIGS. 2B-1, 2B-2, and 2B-3. The results show resolution of these analytes.

O-, m-, and p-xylene were separated using a gradient mixture of solvent A and solvent B according to Table 1. Performance results are shown in FIGS. 2C-1, 2C-2, and 2C-3. The results show resolution of these analytes.

Cis- and trans-2-hydroxycinnamic acid were separated using a gradient mixture of solvent A and solvent B according to Table 1. Performance results are shown in FIG. 2D. The results show resolution of these analytes.

Procainamide and prilocaine were separated using a gradient mixture of solvent A and solvent B according to Table 2. Performance results are shown in FIGS. 2E-1 and 2E-2. The results show resolution of these analytes.

### Example 6 Performance of HPLC Column Loaded with Carbon Microspheres Functionalized with C18

A 2.1×50 mm HPLC test column is prepared containing 3.5 μm nonporous carbon microspheres functionalized with C18 prepared according to the procedure of Example 3 as the stationary phase.

The test column is used to separate mixtures of the several different chemical species. The test column is operated at a temperature of 35° C. Solvent A consists of 50 mL acetonitrile, 950 mL water, and 1 mL formic acid. Solvent B consists of 800 mL acetonitrile, 200 mL water, and 1 mL formic acid. The flow rate is 0.3 mL/min. Detection is at UV 254 nm. Injection is 0.1 μL ca., 0.5 mg/mL in 20% methyl alcohol aqueous.

Methyl paraben and ethyl paraben are separated using an isocratic mixture of 75% solvent A and 25% solvent B. Performance results show clear baseline separation of these analytes.

2-, 3-, and 4-methylhippuric acid are separated using a gradient mixture of solvent A and solvent B according to Table 1. Performance results show resolution of these analytes.

TABLE 1

| Time (min) | % Solvent A | % Solvent B |
|---|---|---|
| 0.00 | 95 | 5 |
| 10.00 | 15 | 85 |
| 10.01 | 95 | 5 |
| 17.00 | 95 | 5 |

O-, m-, and p-xylene are separated using a gradient mixture of solvent A and solvent B according to Table 1. Performance results show resolution of these analytes.

Cis- and trans-2-hydroxycinnamic acid are separated using a gradient mixture of solvent A and solvent B according to Table 1. Performance results show resolution of these analytes.

Procainamide and prilocaine are separated using a gradient mixture of solvent A and solvent B according to Table 2. Performance results show resolution of these analytes.

TABLE 2

| Time (min) | % Solvent A | % Solvent B |
|---|---|---|
| 0.00 | 95 | 5 |
| 10.00 | 50 | 50 |
| 10.01 | 95 | 5 |
| 17.00 | 95 | 5 |

### EMBODIMENTS

Various embodiments are listed below. It will be understood that the embodiments listed below may be combined with all aspects and other embodiments in accordance with the scope of the invention.

Embodiment 1. A chromatographic material comprising carbon microspheres which are surface-derivatized with an organic functional group.

Embodiment 2. The chromatographic material according to Embodiment 1, wherein the carbon microspheres have a particle size less than 10 μm.

Embodiment 3. The chromatographic material according to Embodiment 1, wherein the carbon microspheres have a particle size less than 5 μm.

Embodiment 4. The chromatographic material according to Embodiment 1, wherein the carbon microspheres have a particle size less than 2 μm.

Embodiment 5. The chromatographic material according to Embodiment 1, wherein the carbon microspheres have a particle size less than 1 μm.

Embodiment 6. The chromatographic material according to Embodiment 1, wherein the carbon microspheres have a particle size in the range from about 2 μm to 5 μm.

Embodiment 7. The chromatographic material according to Embodiment 1, wherein the carbon microspheres have a particle size in the range from about 1 μm to 2 μm.

Embodiment 8. The chromatographic material according to Embodiment 1, wherein the carbon microspheres have a particle size greater than 10 μm.

Embodiment 9. The chromatographic material according to any preceding Embodiment, wherein the organic functional group is selected from C1 to C18 alkyl, phenyl, amino, and cyano.

Embodiment 10. The chromatographic material according to any preceding Embodiment, wherein the carbon microspheres are stable at a temperature in the range from about 25° C. to 200° C.

Embodiment 11. The chromatographic material according to any preceding Embodiment, wherein the carbon microspheres are stable at a pH in the range from about 0 to 14.

Embodiment 12. The chromatographic material according to any preceding Embodiment, wherein the carbon microspheres have a surface area in the range of 1 to 400 m²/g.

Embodiment 13. The chromatographic material according to any preceding Embodiment, wherein the carbon microspheres are nonporous.

Embodiment 14. The chromatographic material according to any preceding Embodiment, wherein the carbon microspheres comprise solid carbon.

Embodiment 15. The chromatographic material according to any of Embodiments 1-11 and 13-14, wherein the carbon microspheres comprise a surface area less than 1 m²/g.

Embodiment 16. A chromatographic material comprising nonporous carbon microspheres.

Embodiment 17. The chromatographic material according to Embodiment 16, wherein the carbon microspheres have a particle size less than 10 μm.

Embodiment 18. The chromatographic material according to Embodiment 16, wherein the carbon microspheres have a particle size less than 5 μm.

Embodiment 19. The chromatographic material according to Embodiment 16, wherein the carbon microspheres have a particle size less than 2 μm.

Embodiment 20. The chromatographic material according to Embodiment 16, wherein the carbon microspheres have a particle size less than 1 μm.

Embodiment 21. The chromatographic material according to Embodiment 16, wherein the carbon microspheres have a particle size in the range from about 2 μm to 5 μm.

Embodiment 22. The chromatographic material according to Embodiment 16, wherein the carbon microspheres have a particle size in the range from about 1 μm to 2 μm.

Embodiment 23. The chromatographic material according to Embodiment 16, wherein the carbon microspheres have a particle size greater than 10 μm.

Embodiment 24. The chromatographic material according to any of Embodiments 16-23, wherein the organic functional group is selected from C1 to C18 alkyl, phenyl, amino, and cyano.

Embodiment 25. The chromatographic material according to any of Embodiments 16-24, wherein the carbon microspheres are stable at a temperature in the range from about 25° C. to 200° C.

Embodiment 26. The chromatographic material according to any of Embodiments 16-25, wherein the carbon microspheres are stable at a pH in the range from about 0 to 14.

Embodiment 27. The chromatographic material according to any of Embodiments 16-26, wherein the carbon microspheres comprise solid carbon.

Embodiment 28. The chromatographic material according to any of Embodiments 16-27, wherein the carbon microspheres comprise a surface area less than 1 m²/g.

Embodiment 29. A method of making a carbon microspheres comprising: obtaining a plurality of microspheres consisting of a carbonizable polymeric material; oxidizing the polymeric microspheres in an oxygen-containing atmosphere at a temperature in the range of about 200° C. to 300° C. for a time period in the range of about 4 to 8 hours; carbonizing the microspheres in an oxygen-free atmosphere by heating the microspheres to a temperature in the range of about 800° C. to 1000° C. over a time period of at least about 3 hours; high temperature heating the microspheres in an oxygen-free atmosphere at a temperature greater than about 1200° C. for a time period of at least about 3 hours; and hydrogen treating the microspheres in an atmosphere comprising hydrogen (H₂) at a temperature in the range of about 900° C. to 1100° C. for a time period of at least about 1 hour to form a plurality of carbon microspheres.

Embodiment 30. The method according to Embodiment 29, wherein the carbonizable polymeric material comprises polystyrene divinylbenzene.

Embodiment 31. The method according to Embodiment 29, wherein the carbonizable polymeric material comprises non-porous polystyrene divinylbenzene.

Embodiment 32. The method according to any of Embodiments 29-31, wherein the carbonizing oxygen-free atmosphere comprises argon.

Embodiment 33. The method according to any of Embodiments 29-31, wherein the carbonizing oxygen-free atmosphere comprises nitrogen.

Embodiment 34. The method according to any of Embodiments 29-33, wherein the carbonizing is performed over a time period of at least about 4 hours.

Embodiment 35. The method according to any of Embodiments 29-34, wherein the microspheres are heated during the carbonizing step at a rate less than 5° C./min.

Embodiment 36. The method according to any of Embodiments 29-35, wherein the high temperature heating oxygen-free atmosphere comprises argon.

Embodiment 37. The method according to any of Embodiments 29-35, wherein the high temperature heating oxygen-free atmosphere comprises nitrogen.

Embodiment 38. The method according to any of Embodiments 29-37, wherein the high temperature heating is performed for a time period of at least about 4 hours.

Embodiment 39. The method according to any of Embodiments 29-38, wherein the high temperature heating occurs at a temperature in the range of about 1200° C. to 2500° C.

Embodiment 40. The method according to any of Embodiments 29-38, wherein the high temperature heating occurs at a temperature in the range of about 1500° C. to 2500° C.

Embodiment 41. The method according to any of Embodiments 29-38, wherein the high temperature heating occurs at a temperature in the range of about 2000° C. to 2500° C.

Embodiment 42. The method according to any of Embodiments 29-41, wherein the hydrogen heating is performed for a time period of at least 2 hours.

Embodiment 43. The method according to any of Embodiments 29-42, further comprising bonding an organic functional group to a surface of the carbon microspheres.

Embodiment 44. The method according to Embodiment 43, wherein the organic functional group is selected to provide enhanced chromatographic selectivity, enhanced chromatographic column chemical stability, enhanced chromatographic column efficiency, and/or enhanced mechanical strength.

Embodiment 45. The method according to any of Embodiments 43-44, wherein the organic functional group is selected from C1 to C18 alkyl, phenyl, amino, and cyano.

Embodiment 46. The method according to any of Embodiments 43-45, wherein the organic functional group is bonded to the surface of the carbon microspheres by reacting the surface of the carbon microspheres with an iodide form of the organic functional group in the presence of lithium or sodium in ammonia.

Embodiment 47. A method of separating a compound of interest from a mixture, the method comprising: (a) providing the mixture containing the compound of interest; (b) introducing a portion of the mixture to a chromatographic system having a chromatographic column; and (c) eluting the separated compound of interest from the chromatographic column; wherein the chromatographic column has a stationary phase comprising the chromatographic material according to any of Embodiments 1-27.

Reference throughout this specification to “one embodiment,” “certain embodiments,” “one or more embodiments” or “an embodiment” means that a particular feature, structure, material, or characteristic described in connection with the embodiment is included in at least one embodiment of the invention. Thus, the appearances of the phrases such as “in one or more embodiments,” “in certain embodiments,” “in one embodiment” or “in an embodiment” in various places throughout this specification are not necessarily referring to the same embodiment of the invention. Furthermore, the particular features, structures, materials, or characteristics may be combined in any suitable manner in one or more embodiments. Although specific terms are employed herein, they are used in a generic and descriptive sense only, and not for purposes of limitation.

While the invention herein has been described with reference to particular embodiments, it is to be understood that these embodiments are merely illustrative of the principles and applications of the present invention. It will be apparent to those skilled in the art that various modifications and variations can be made to the method and apparatus of the present invention without departing from the spirit and scope of the invention. Thus, it is intended that the present invention include modifications and variations that are within the scope of the appended claims and their equivalents.

1. A chromatographic material comprising carbon microspheres which are surface-derivatized with an organic functional group.

2. The chromatographic material according to claim 1, wherein the carbon microspheres have a particle size in the range from about 1 μm to 5 μm.

3. The chromatographic material according to claim 1, wherein the organic functional group is selected from C1 to C18 alkyl, phenyl, amino, and cyano.

4. The chromatographic material according to claim 1, wherein the carbon microspheres are stable at a temperature in the range from about 25° C. to 200° C.

5. The chromatographic material according to claim 1, wherein the carbon microspheres are stable at a pH in the range from about 0 to 14.

6. The chromatographic material according to claim 1, wherein the carbon microspheres have a surface area in the range of 1 to 400 m²/g.

7. The chromatographic material according to claim 1, wherein the carbon microspheres comprise a surface area less than 1 m²/g.

8. The chromatographic material according to claim 1, wherein the carbon microspheres are nonporous.

9. A chromatographic material comprising nonporous carbon microspheres.

10. The chromatographic material according to claim 9, wherein the organic functional group is selected from C1 to C18 alkyl, phenyl, amino, and cyano.

11. The chromatographic material according to claim 9, wherein the carbon microspheres comprise solid carbon.

12. The chromatographic material according to claim 9, wherein the carbon microspheres comprise a surface area less than 1 m²/g.

13. A method of making carbon microspheres comprising:
obtaining a plurality of microspheres consisting of a carbonizable polymeric material;
oxidizing the polymeric microspheres in an oxygen-containing atmosphere at a temperature in the range of about 200° C. to 300° C. for a time period in the range of about 4 to 8 hours;
carbonizing the microspheres in an oxygen-free atmosphere by heating the microspheres to a temperature in the range of about 800° C. to 1000° C. over a time period of at least about 3 hours;
high temperature heating the microspheres in an oxygen-free atmosphere at a temperature greater than about 1200° C. for a time period of at least about 3 hours; and
hydrogen treating the microspheres in an atmosphere comprising hydrogen (H₂) at a temperature in the range of about 900° C. to 1100° C. for a time period of at least about 1 hour to form a plurality of carbon microspheres.

14. The method according to claim 13, wherein the carbonizable polymeric material comprises polystyrene divinylbenzene.

15. The method according to claim 13, wherein the high temperature heating is performed for a time period of at least 4 hours.

16. The method according to claim 13, wherein the high temperature heating occurs at a temperature in the range of about 1200° C. to 2500° C.

17. The method according to claim 13, wherein the hydrogen heating is performed for a time period of at least 2 hours.

18. The method according to claim 13, further comprising bonding an organic functional group to a surface of the carbon microspheres.

19. The method according to claim 13, wherein the organic functional group is bonded to the surface of the carbon microspheres by reacting the surface of the carbon microspheres with an iodide form of the organic functional group in the presence of lithium or sodium in ammonia.

20. A method of separating a compound of interest from a mixture of different compounds, the method comprising:
(a) providing the mixture containing the compound of interest;
(b) introducing a portion of the mixture to a chromatographic system having a chromatographic column; and
(c) eluting the separated compound of interest from the chromatographic column;
wherein the chromatographic column has a stationary phase comprising the chromatographic material according to claim 1.
